(19) United States
(12) Patent Application Publication
(10) Pub. No.: US 2025/0121084 A1
(43) Pub. Date: Apr. 17, 2025

(54) ANTI-CLAUDIN-6 CONJUGATES

(71) Applicant: ADC THERAPEUTICS SA, Epalinges (CH)

(72) Inventors: Paul HOGG, London (GB); Nicolas VEILLARD, London (GB); Patricius Hendrikus Cornelis VAN BERKEL, London (GB)

(21) Appl. No.: 18/971,848

(22) Filed: Dec. 6, 2024

Related U.S. Application Data
(63) Continuation of application No. PCT/EP2024/079218, filed on Oct. 16, 2024.

(30) Foreign Application Priority Data
Oct. 17, 2023 (EP) 23204138.4

(51) Int. Cl. A61K 47/68; A61P 35/00; C07K 16/28

(52) U.S. Cl. CPC A61K 47/68037; A61K 47/6849; A61K 47/6889; A61P 35/00; C07K 16/28

(57) ABSTRACT

This disclosure relates to antibody conjugates comprising an antibody that binds specifically to the Claudin-6 protein, conjugated to an Exatecan, and associated therapeutic uses.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 22 KB file named “0233-0036US1_SL.xml,” created on Dec. 2, 2024.

### FIELD OF THE INVENTION

The present disclosure relates to cell binding agent conjugates and treatment of diseases and disorders.

### BACKGROUND

Ovarian cancer (OC) is a common gynecological malignancy and is a highly heterogeneous epithelial tumor with different histological subtypes as well as genetic and biological features, including serous carcinoma, endometrioid carcinoma, clear Cellular and mucinous carcinomas. Every year, there are 310,000 new cases of ovarian cancer and more than 200,000 deaths worldwide (5). 75% of ovarian cancers are serous cancers, with a five-year survival rate of 35%. They are a very malignant type of cancer and are usually diagnosed at an advanced stage. This class of tumors is remarkably aggressive, and in terms of targeted drugs, in addition to anti-angiogenic therapy, which has been clinically shown to improve progression-free survival in patients with stage III-IV serous ovarian cancer, advanced serous ovarian cancer initially Sensitive to platinum-based chemotherapy but relapses shortly after initial response, the primary treatment in approximately 70-80% of cases is surgery.

The Claudin (CLDN) family of proteins are transmembrane proteins located at the tight junction between epithelial cells and endothelial cells. The distribution of CLDN family proteins is specific to tissues and organs, and their main functions are intercellular adhesion, maintenance of cell polarity, regulation of paracellular permeability, and participation in the regulation of cell proliferation and differentiation. Recent studies have shown that the expression of some members of the CLDN family is up-regulated during carcinogenesis, and ectopic activation occurs in tissues that are not normally distributed in the case of carcinogenesis.

A large number of research data in TCGA showed that the level of CLDN6 in ovarian cancer patients was significantly up-regulated compared with normal ovarian tissue. =426). CLDN6 is only highly expressed during embryonic development and not expressed in normal adult tissues. The highest expression of CLDN6 in normal adult tissues is the testis, and the expression level is only 0.83 TPM. In addition to high expression in ovarian cancer, CLDN6 is also highly expressed in testicular cancer (159.9 TPM, n=137), uterine sarcoma (8.4 TPM, n=57), and part of endometrial cancer, gastric cancer, and lung cancer. Studies have shown that high expression of CLDN6 in endometrial cancer is associated with multiple clinicopathological factors and is an independent prognostic factor. Kaplan-Meier analysis showed a significant difference in overall survival and recurrence-free survival between the high and low CLDN6 expression groups, with the 5-year survival rate of about 30% in the high CLDN6 expression group compared to the low 5-year survival rate in the low expression group. 89%. In addition to endometrial cancer, high expression of CLDN6 is also negatively correlated with the prognosis of gastric cancer and urothelial cancer. The lack of expression in normal tissues, high expression in tumor tissues, and negative correlation with tumor prognosis make CLDN6 a good tumor target.

CLDN6 protein is a four-transmembrane protein with four transmembrane hydrophobic regions and two extracellular loops. Its recombinant protein is extremely difficult to express, so there is no suitable protein antigen for immunization, which brings difficulties to the immunization and screening of antibodies against CLDN6. In addition, the CLDN family proteins have high homology. When targeting CLDN6, it is necessary to avoid binding to CLDN3 and CLDN4, which are widely expressed in normal tissues and have high homology to CLDN6, so as to avoid possible cross-binding. Toxicity issue. The above are the two major difficulties in the development of anti-CLDN6 antibodies.

Accordingly, a need exists for anti-cancer therapies that target the biological activities of Claudin-6.

### SUMMARY

The present disclosure is directed to an anti-Claudin-6 antibody conjugated to an Exatecan and its use in therapy.

Accordingly in a first aspect is provided an antibody drug conjugate of formula (I):

Ab-L-Dp (I)

wherein:

Ab is an antibody that binds to Claudin-6, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;

L-Dp is a drug linker conjugated to the Ab, such as via cysteine residues, wherein L typically comprises a polyglycine moiety, Glyₙ wherein n is from 5 to 8, and D is an Exatecan, wherein the drug linker can be cleaved under cellular conditions to release an Exatecan with following formula:

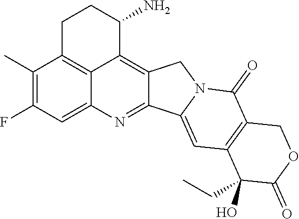 [SMILES: CC[C@@]1(O)C(=O)OCc2c1cc1n(c2=O)Cc2c-1nc1cc(F)c(C)c3c1c2[C@@H](N)CC3]

The drug loading is represented by p, the number of drug units per antibody. Drug loading may typically range from 1 to 8 Drug units (D) per antibody, such as from 1 to 6. For compositions, p represents the average drug loading of the conjugates in the composition, and p ranges from 1 to 8. In one embodiment, p ranges from 1 to 6, for example as a result of the removal/substitution of one or more cysteines, such as hinge region cysteines, in the antibody.

In a second aspect is provided the conjugate of the first aspect together with one or more pharmaceutically acceptable carriers or diluents, such as a pharmaceutical composition in liquid or lyophilized form. The composition may comprise a therapeutically effective amount of a chemotherapeutic agent.

The conjugate or compositions comprising it may be used in therapy. For example, in a method of treating a proliferative disease, the method comprising administering an effective amount of the conjugate or the composition to an individual in need of such treatment.

The treated proliferative disease may be cancer, such as ovarian cancer, non-small cell lung carcinoma (NSCLC), gastric cancer, oesophageal cancer, endometrial cancer or hepatocellular carcinoma (HCC).

The proliferative disease may be characterised by the presence of a neoplasm comprising both Claudin-6+ve and Claudin-6−ve cells, and/or may be a solid tumour. The proliferative disease may be characterised by the over-expression of Claudin-6, either in all or most of the aberrant cells.

### DETAILED DESCRIPTION

### Antibody Drug Conjugates

In some embodiments, the present disclosure provides, inter alia, an antibody drug conjugate comprising (i) a cell binding agent which comprises the complementarity determining regions of the GB01 antibody described herein such that the cell binding agent binds to Claudin-6 on the surface of cell; (ii) a cytotoxin comprising Extecan. The cytotoxin is connected to the cell binding agent via one or more linkers—examples of suitable linkers are described in more detail below. In one embodiment each linker is connected through the NH₂ on the F ring of Exatecan as described below.

Various aspects and embodiments of the disclosures herein are thus suitable for use in providing an Exatecan to a preferred site in a subject. The conjugate in some embodiments preferably allows the release of an active Exatecan that does not retain any part of the linker.

Before transport or delivery into a cell, the antibody-drug conjugate (ADC) is preferably stable and remains intact, i.e. the antibody remains linked to the drug moiety. The linkers in such preferred embodiments are stable outside the target cell and may be cleaved at some efficacious rate inside the cell. An effective linker in some embodiments will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the Exatecan drug moiety. Stability of the ADC may be measured by standard analytical techniques such as mass spectroscopy, HPLC, and the separation/analysis technique LC/MS.

### Cell Binding Agents/Antibodies

The cell binding agent in some embodiments is an antibody that binds to Claudin-6. In some specific embodiments the cell binding agent is an antibody that binds to Claudin-6 and which comprises the complementarity determining regions (CDRs) of monoclonal antibody GB01 (SEQ ID NOs 9 and 11).

The term “antibody” herein is used in the broadest sense and specifically covers monoclonal antibodies, including both intact antibodies and antibody fragments, so long as they exhibit the desired biological activity, for example, the ability to bind Claudin-6. Antibodies may be murine, rat, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule.

A variety of immunoglobulin variant formats are known in the art which are derived from conventional immunoglobulins, such as bispecific antibodies, scFvs, nanobodies and the like. These are all within the scope of the term “antibody” provided they retain the GB01 CDRs and/or Claudin-6 binding activity.

Thus in some embodiments the antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8.

The CDR sequences as disclosed herein have been identified and defined using the Kabat numbering scheme (Kabat et al., U.S. Department of Health and Human Services, 1991).

In one embodiment the antibody comprises a VH domain having the sequence according to SEQ ID NO. 1. In another embodiment the antibody comprises a VL domain having the sequence according to SEQ ID NO. 2. Thus the antibody may comprise a VH domain and a VL domain where the VH comprises the sequence of SEQ ID NO.1 and the VL domain comprises the sequence of SEQ ID NO.2.

The VH and VL domain(s) in various embodiments form an antibody antigen binding site that binds Claudin-6.

In some embodiments the antibody is an intact antibody comprising a VH domain and a VL domain, the VH and VL domains having sequences of SEQ ID NO.1 paired with SEQ ID NO.2.

In one embodiment the light chain is a human kappa light chain.

In one embodiment, the antibody that binds to Claudin-6 comprises a heavy chain with the amino acid sequence shown in SEQ ID NO: 9 or SEQ ID NO: 10 and a light chain with the amino acid sequence shown in SEQ ID NO: 11 or SEQ ID NO: 12.

As used herein, “binds Claudin-6” is used to mean the cell binding agent or antibody binds Claudin-6 with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds Claudin-6 with an association constant (Kₐ) at least 2, 3, 4, 5, 10, 20, 50, 100, 200, 500, 1000, 2000, 5000, 104, 105 or 106-fold higher than the antibody's association constant for BSA, when measured at physiological conditions. The cell binding agents or antibodies of the disclosure can in some embodiments bind Claudin-6 with a high affinity. For example, in some embodiments the antibody can bind Claudin-6 with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

As used herein, Claudin-6 refers to the amino acid sequence disclosed at UniProt accession no: UniProt: P567047 (entry version 178) or a fragment thereof, such as an extracellular domain. In some embodiments, the Claudin-6 polypeptide corresponds to an amino acid sequence having at least 70%, 80%, 90%, 95%, 99% or 100% sequence identity with the full-length of the amino acid sequence disclosed at UniProt accession no: UniProt: P56747 (entry version 178) or a fragment thereof, such as an extracellular domain.

“Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method or may be made by recombinant DNA methods. The monoclonal antibodies may also be isolated from phage antibody libraries or from transgenic mice carrying a fully human immunoglobulin system.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567). Chimeric antibodies include “primatized” antibodies comprising variable domain antigen-binding sequences derived from a non-human primate and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

### Modification of Antibodies

The antibodies disclosed herein may be modified. For example, to make them less immunogenic to a human subject. This may be achieved using any of a number of techniques familiar to the person skilled in the art. Such techniques includes humanisation to reduce the in vivo immunogenicity of a non-human antibody or antibody fragment. There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

Other sequence modification can be made to assist with conjugation of drugs or other substances of interest to particular sites in the antibody or to regulate the drug to antibody ratio. For example one or more cysteine residues, such as in the hinge region, may be substituted or introduced, where conjugation to a cysteine residue is desired.

In one embodiment, the antibody has one or more cysteine residues introduced outside the hinge region, e.g. to take advantage of the THIOMAB™ site-specific approach (see Adhikari, P., Zacharias, N., Ohri, R., Sadowsky, J. (2020). Site-Specific Conjugation to Cys-Engineered THIOMAB™ Antibodies. In: Tumey, L. (eds) Antibody-Drug Conjugates. Methods in Molecular Biology, vol 2078. Humana, New York, NY; and Junutula et al., 2008, Site-specific conjugation of a cytotoxic drug to an antibody improves the therapeutic index. Nat Biotechnol 26 (8): 925-932. A particular example, used herein, is a substitution at amino acid residue 205 (EU numbering) of the IgG light chain, e.g. a V205C mutation, as shown in SEQ ID NO: 12 (amino acid residue 211).

In one embodiment, a heavy chain hinge region cysteine is mutated to remove a potential site for conjugation. A wild-type antibody typically has 8 surface-exposed cysteine residues available for conjugation—three in each of the heavy chain hinges (C220, C226 and C229—EU numbering corresponding to residues 222, 228 and 231 respectively of SEQ ID NOs: 9 and 10) and one at the C-terminus of each of the light chains (C214 kappa or C213 lambda—EU numbering). In a particular embodiment, C226 in the heavy chain (residue 225 in SEQ ID NO: 9) is mutated, such as substituted or deleted.

The amino acid substituted for a cysteine residue can, for example, be selected from valine, serine, threonine, alanine, glycine, leucine and isoleucine. In one embodiment the amino acid substitution is a valine for the interchain cysteine residue as shown in SEQ NO: 10.

Other modifications include those in the Fc region that reduce effector functions, such as Fc gamma binding (Wang et al., 2018, Protein Cell 9 (1): 63-73). For example, the sequence may include a PALALA series of mutations (L234A/L235A (Xu et al., 2000, Cell. Immunol. 200:16-26); and P329A (Michaelsen et al., 2009, Scand. J. Immunol. 70 (6): 553-64)—EU numbering corresponding to residue numbers 233, 234 and 328 of SEQ ID NO: 9). Another example is an FES mutation (see Oganesyan et al., 2008, Acta Crystallographica, D64:700-704).

In one embodiment the antibody has an Fc region as shown in SEQ ID NO: 10.

### Exatecan Warhead

Exatecan is a topoisomerase inhibitor. It is a more extensively modified derivative of camptothecin, with an additional alicyclic ring fused to rings A and B that bears a solubilising primary amine. There are also lipophilic substituents at positions 10 and 11 on ring A that may help to enhance membrane permeability. It is about 2.5-fold more potent than SN38 (the active metabolite of irinotecan) at stabilizing the topo I/DNA complex, and as a cytotoxin in a variety of cell lines.

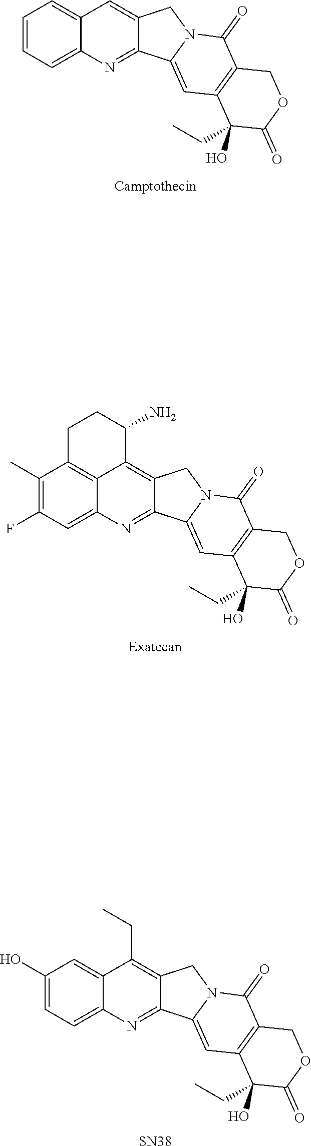 [SMILES: CC[C@@]1(O)C(=O)OCc2c1cc1n(c2=O)Cc2cc3ccccc3nc2-1 ; CC[C@@]1(O)C(=O)OCc2c1cc1n(c2=O)Cc2c-1nc1cc(F)c(C)c3c1c2[C@@H](N)CC3 ; CCc1c2c(nc3ccc(O)cc13)-c1cc3c(c(=O)n1C2)COC(=O)[C@]3(O)CC ; "SN38"]

Exatecan (also known as DX-8951) is available from a number of commercial sources e.g. as Exatecan mesylate (CAS No.: 169869-90-3).

### Drug Linkers

A wide variety of linker technologies are available in the art to link cytotoxins to cell binding agents. Linkers can incorporate various different moieties to assist with antibody-drug conjugate stability and determine drug release characteristics. For example the linker may include a cleavable moiety, such as one that is cleavable by cathepsin B (e.g. Valine-Alanine or Valine-Citrulline) or (ii) a GGFG motif (SEQ ID NO: 15), which is also cleaved by lysosomal proteases. Another strategy is to use a pH-sensitive linker whereby the lower pH of the endosome and lysosome compartments the hydrolysis of an acid-labile group within the linker, such as a hydrazone. Alternative a linker may be non-cleavable, which can avoid or reduce off-target effects and improve plasma stability during circulation.

The functionality that allows conjugation to the cell binding agent is based on the site of conjugation and its chemistry. For conjugation to cysteines, thiol-reactive maleimide is the most applied reactive handle, although it is also possible to create a disulfide bridge by oxidation with a linker bearing a sulfhydryl group. Aldehyde or keto functional groups such as oxidized sugar groups or pAcPhe unnatural amino acids can be reacted with hydrazides and alkoxyamines to yield acid-labile hydrazones or oxime bonds. In addition, a hydrazine can be coupled with an aldehyde via HIPS ligation to generate a stable C—C linkage.

In some embodiments, the antibody drug conjugates of the disclosure can be described as Ab-L-D, where Ab is the anti-Claudin-6 antibody, D is Exatecan and L is a linker. The number of Drug moieties per Ab (the drug loading, p) depends on the number of linkers attached to each Ab, and the number of Drug moieties per linker. Typically the drug loading, p, is from 1 to 8, such as from 1 to 2, 1 to 4, 1 to 6, 2 to 6 or 3 to 6, such as from 3 to 6. Drug loading is typically considered on an average basis since variations can arise from the conjugation process (a composition comprising a plurality of antibody drug conjugate molecules will typically have individual molecules with from zero to the maximum number of drug molecules possible). Methods for determining average drug loading are known in the art.

The linker comprises a polyglycine stretch, Glyₙ where n is from 5 to 8 glycines (SEQ NOs: 16 to 20), which we have shown is beneficial in reducing aggregation. The glycines are continuous e.g. Gly-Gly-Gly-Gly-Gly (SEQ ID NO: 16), with no intervening amino acids. In one embodiment n is 5 or 6, such as 5.

The drug linker is designed so that under cellular conditions, the Exatecan warhead is released without any remaining portions of the linker (e.g. by the use of self-immolative linker chemistry, such as para-aminobenzylcarbamate—PABC).

In one embodiment the linker is of formula (Ia): -G^{LL}-X-Glyₙ-A-
wherein G^{LL} is for connection of the linker to a cysteine residue on the antibody; X is an optional spacer portion; n is from 5 to 8; and A comprises a moiety cleavable under cellular conditions and optionally a self-immolative moiety.

### G^{LL}

G^{LL} may be selected from:

| | |
|---|---|
| (G^{LL1-1}) | 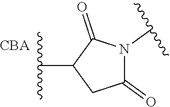 [SMILES: *C1CC(=O)N(*)C1=O] |
| (G^{LL1-2}) | 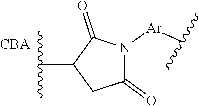 |
| (G^{LL2}) | 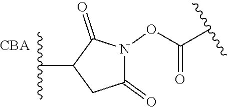 [SMILES: *C(=O)ON1C(=O)CC(*)C1=O] |
| (G^{LL3-1}) | 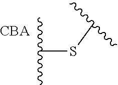 [SMILES: *S*] |
| (G^{LL3-2}) | 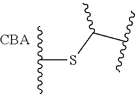 [SMILES: *SC(*)C] |
| (G^{LL-4}) | 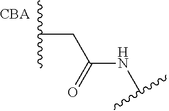 [SMILES: *CC(=O)N*] |
| (G^{LL5}) | 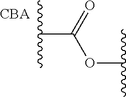 [SMILES: *OC(*)=O] |
| (G^{LL6}) | 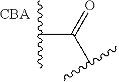 [SMILES: *C(*)=O] |
| (G^{LL7}) | 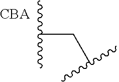 [SMILES: *C*] |
| (G^{LL8}) | 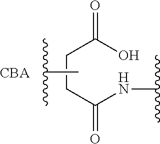 |

where Ar represents a C₅₋₆ arylene group, e.g. phenylene and X represents C₁₋₅ alkyl. CBA indicates the end of G^{LL} connected to the antibody.

In some embodiments, G^{LL} is selected from G^{LL1-1} and G^{LL1-2}. In some of these embodiments, G^{LL} is G^{LL1-1}.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”. Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₅ alkyl: The term “C₁₋₅ alkyl” as used herein, pertains to a monovalent moiety obtained by removing a hydrogen atom from a carbon atom of a hydrocarbon compound having from 1 to 5 carbon atoms, which may be aliphatic or alicyclic, and which may be saturated or unsaturated (e.g. partially unsaturated, fully unsaturated). The term “C₁₋₅ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

### X

X is an optional linker portion connecting G^{LL} to the polyglycine stretch. Where X is present, X may for example be selected from —(CR¹R²) y-, —(CR¹R²) y-C(O)—, and —C(O)—(CR¹R²) y-C(O)—, wherein y is an integer in the range 0 to 6, wherein one or more CR¹R² may be optionally replaced by NR¹; R¹ and R² are independently selected from H, R^{2a}, C(O)OH and C(O)R^{2a}, wherein R^{2a} is selected from optionally substituted C₁-C₆ (hetero)alkyl groups, C₃-C₁₀ (hetero)cycloalkyl groups, C₆-C₁₀ (hetero) aryl groups, C₇-C₁₄ alkyl(hetero) aryl groups and C₇-C₁₄ (hetero) arylalkyl groups; wherein R^{3a} is independently selected from hydrogen and C₁-C₄ alkyl groups.

In one embodiment, y is an integer in the range 1 to 6, such as from 1 to 5, 1 to 4, 1 to 3, or 1 or 2.

In one embodiment R¹ and R² are both H.

In one embodiment, X is —(CH₂)₁₋₅—C(O)—. In a particular embodiment X is —CH₂C(O)— or —CH₂CH₂C(O)—.

### A

Linker portion A connects the polyglycine stretch to the Exatecan and includes components that allow for cleavage of the linker under cellular conditions e.g. cellular protease recognition motifs such as a cleavage site for cathepsin.

For example A may comprise a dipeptide selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O}, ^{NH}-Phe-Cit-^{C═O}, ^{NH}-Leu-Cit-^{C═O}, ^{NH}-IIe-Cit-^{C═O}, ^{NH}-Phe-Arg-^{C═O}, ^{NH}-Trp-Cit-^{C═O}, and ^{NH}-Gly-Val-^{C═O};
   where Cit is citrulline.

In one embodiment the dipeptide is Val-Ala or Val-Cit, such as Val-Ala.

In the above representations of dipeptide residues, NH-represents the N-terminus, and —C═O represents the C-terminus of the residue. The N-terminus is orientated towards the polyglycine stretch.

The self-immolative moiety which allows for the generation of a free amine on the Exatecan F ring is typically p-aminobenzylcarbamate (PABC) or a variant thereof. Accordingly in one embodiment, A is Val-Cit-PABC or Val-Ala-PABC.

In one embodiment the linker is of formula (IIa):

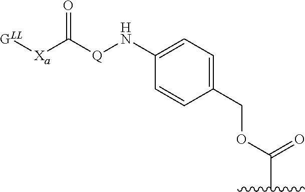

wherein G^{LL} is as defined above, and Xₐ is selected from —(CR¹R²)y- and —C(O)—(CR¹R²)y-, wherein y is an integer in the range 0 to 6, wherein one or more CR¹R² may be optionally replaced by NR¹; R¹ and R² are independently selected from H, R^{2a}, C(O)OH and C(O)R^{2a}, wherein R^{2a} is selected from optionally substituted C₁-C₆ (hetero)alkyl groups, C₃-C₁₀ (hetero)cycloalkyl groups, C₆-C₁₀ (hetero)aryl groups, C₇-C₁₄ alkyl(hetero)aryl groups and C₇-C₁₄ (hetero)arylalkyl groups; wherein R^{3a} is independently selected from hydrogen and C₁-C₄ alkyl groups.

In one embodiment, y is an integer in the range 1 to 6, such as from 1 to 5, 1 to 4, 1 to 3, or 1 or 2.

In one embodiment R¹ and R² are both H.

In one embodiment, Xₐ is —(CH₂)₁₋₅—. In a particular embodiment Xₐ is —CH₂— or —CH₂CH₂—.

In a particular embodiment G^{LL} is G^{LL1-1}

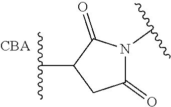 [SMILES: *C1CC(=O)N(*)C1=O]

Q is -Glyₙ-AA₁-AA₂ where n is from 5 to 8, such as 5 or 6 and AA₁-AA₂ is a dipeptide residue which is a recognition site for cathepsin such that the linker is susceptible to cathepsin-mediated cleavage.

In one embodiment, AA₁-AA₂ is selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O}, ^{NH}-Phe-Cit-^{C═O}, ^{NH}-Leu-Cit-^{C═O}, ^{NH}-Ile-Cit-^{C═O}, ^{NH}-Phe-Arg-^{C═O}, ^{NH}-Trp-Clt-^{C═O}, and ^{NH}-Gly-Val-^{C═O};
   where Cit is citrulline.

In one embodiment the dipeptide is Val-Ala or Val-Cit, such as Val-Ala.

In the above representations of dipeptide residues, NH— represents the N-terminus, and —C═O represents the C-terminus of the residue. The C-terminus binds to the NH attached to the benzene ring.

The linker is typically connected to the Exatecan via the NH₂ on the F ring.

### Drug Linker Embodiments

In some embodiments, the drug linker, L-D, is

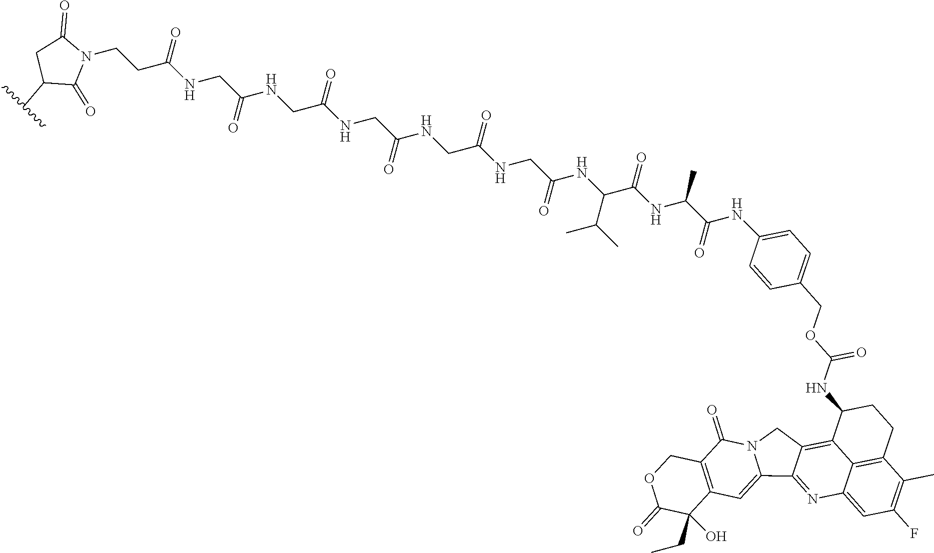 [SMILES: *C1CC(=O)N(CCC(=O)NCC(=O)NCC(=O)NCC(=O)NCC(=O)NCC(=O)NC(C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

A preferred antibody drug conjugate has the drug linker above linked to a Claudin-6 antibody with a heavy chain as shown in SEQ ID NOs: 9 or 10 and a light chain as shown in SEQ ID NO: 11.

### Site of Conjugation and Drug Loading

Drug linkers can be conjugated to a cell binding agent, such as an antibody, using a variety of methods known in the art and at a number of different sites. Conjugation sites are typically cysteine residues in the antibody sequence (endogenous or engineered). With respect to cysteine conjugation, in one embodiment the cysteine is an endogenous cysteine located in the hinge region or Fc domain or a cysteine in the constant region of the light chain (for example C220, C226 or C229 of the heavy chain in IgG1 or the equivalent; or C214 (kappa) or C213 (lambda) of the light chain (EU numbering)). In another embodiment the cysteine is an engineered cysteine introduced in the hinge region or constant region of the light chain or heavy chain. However other conjugation methods may be used such as conjugation to N-linked glycans (which may be enzymatically trimmed) e.g. using GlycoConnect™ or a microbial transglutaminase-based approach based on conjugation to an endogenous or engineered glutamine residue.

The drug loading is the average number of Exatecan drugs per cell binding agent, e.g. antibody, in a composition comprising a plurality of molecules.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. Only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody, and which do not form intrachain or intermolecular disulfide linkages (Junutula, et al., 2008b Nature Biotech., 26(8):925-932; Dornan et al. (2009) Blood 114(13):2721-2729; U.S. Pat. Nos. 7,521,541; 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the Exatecan drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. This THIOMAB™ approach allows for site-specific conjugation with more homogenous DARs. In one embodiment, a single engineered cysteine residue is introduced and the resulting maximum DAR is 2 (in a composition comprising a mixture, p is from 1.5 to 2).

Conversely, one or more of the endogenous cysteine residues may be mutated to reduce the number of available conjugation sites (as described in the “Modification of antibodies” section above). Since p>5 or 6 can lead to undesirable aggregation, where all four hinge cysteines are available (a total of 8), it can be necessary to tailor the conjugation process to avoid high levels of conjugation as this can lead to significant numbers of species where p=7 or 8. However, avoiding this results in a lower overall average drug to antibody ratio (e.g. around 4). To push the DAR higher whilst avoiding high p ratios can be achieved according to the present disclosure by mutating one of the hinge cysteine residues to leave 2 available conjugation sites per heavy chain and one per light chain—6 in total. In this way a DAR closer to 6 can be achieved whilst avoiding species where p=7 or 8 which may have a tendency to aggregate.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more Exatecan drug moieties and where the drug moieties may be attached to the antibody at various amino acid residues.

In one embodiment, the average number of Exatecan groups per antibody is in the range 1 to 8. In some embodiments the range is selected from 1 to 2, 1 to 4, 1 to 6, 2 to 6 or 3 to 6, such as from 1 to 2 or 3 to 6.

### Preparation of Drug Conjugates

The drug linker may be synthesised as described in, for example, the experimental section below.

The antibody drug conjugates of the present disclosure may then be prepared by conjugating the drug-linker to the antibody via an endogenous or engineered cysteine residue via maleimide, as for example described in U.S. Pat. No. 9,889,207 or Flynn et al., 2016. Mol Cancer Ther 15: 2709.

### Treated Disorders

The therapies described herein include those with utility for anti-cancer activity. In particular, in certain aspects the therapies include an antibody conjugated, i.e. covalently attached by a linker, to an Exatecan drug moiety, i.e. toxin. When the drug is not conjugated to an antibody, the Exatecan drug has a cytotoxic effect. The biological activity of the Exatecan drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides therapies comprising administering a conjugate compound as described herein, which binds to Claudin-6, for use in therapy, wherein the method comprises selecting a subject based on expression of Claudin-6 protein.

In one aspect, the present disclosure provides a therapy with a label that specifies that the therapy is suitable for use with a subject determined to be suitable for such use. The label may specify that the therapy is suitable for use in a subject has expression of Claudin-6 e.g. is a Claudin-6+ve cancer. The label may specify that the subject has a particular type of cancer.

The label may specify that the subject has a Claudin-6+ve cancer.

The range of disorders that may be treated by such therapies is described in more detail below.

In a further aspect there is also provided a therapy as described herein for use in the treatment of a proliferative disease. Another aspect of the present disclosure provides the use of a conjugate compound as described herein in the manufacture of a medicament for treating a proliferative disease.

One of ordinary skill in the art is readily able to determine whether or not a candidate therapy treats a proliferative condition for any particular cell type. For example, assays which may conveniently be used to assess the activity offered by a particular compound are described below.

The therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

The proliferative disease may be characterised by the presence of a neoplasm comprising both Claudin-6+ve and Claudin-6−ve cells.

The target neoplasm or neoplastic cells may be all or part of a solid tumour, such as an advanced solid tumour.

Thus in one embodiment the neoplasm/cancer is itself essentially Claudin-6+ve.

Examples of proliferative conditions that may be treated with the conjugate compounds described herein include, but are not limited to, benign, pre-malignant, and malignant cellular proliferation, including but not limited to, neoplasms, tumours and cancers.

Particular proliferative conditions to be treated include ovarian cancer, lung cancer (such as non-small cell lung cancer), oesophageal cancer, liver cancer such as hepatocellular carcinoma (HCC), endometrial cancer, gastric cancer, testicular cancer, pancreatic cancer, bronchial cancer, breast cancer, ear nose and throat (ENT) cancer, colon cancer, head and neck cancer (such as head and neck squamous cell cancer (HNSCC)), gallbladder cancer, gliomas (such as adult high grade gliomas) and atypical teratoid rhabdoid tumors, and their metastases (e.g. gastric cancer metastasis such as Krukenberg tumor, peritoneal metastasis or lymph node metastasis). In one embodiment the proliferative condition is selected from ovarian cancer and lung cancer (such as non-small cell lung cancer).

### Patient Selection

In certain aspects, the individuals are selected as suitable for treatment with the treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. Typically the individual is an animal or human subject.

In some aspects, individuals are selected on the basis of the amount or pattern of expression of a first target protein. In some aspects, the selection is based on expression of a first target protein at the cell surface.

In some cases, individuals are selected on the basis they have, or are suspected of having, are at risk of having cancer, or have received a diagnosis of a proliferative disease characterised by the presence of a neoplasm comprising cells having a high level of surface expression of Claudin-6. The neoplasm may be composed of cells having a high level of surface expression of Claudin-6. In some cases, high levels of surface expression means that mean number of anti-Claudin-6 antibodies bound per neoplastic cell is greater than 70000, such as greater than 80000, greater than 90000, greater than 100000, greater than 110000, greater than 120000, greater than 130000, greater than 140000, or greater than 150000.

In some cases, individuals are selected on the basis they have, or are suspected of having, are at risk of having cancer, or have received a diagnosis of a proliferative disease characterised by the presence of a neoplasm comprising cells having a low level of surface expression of Claudin-6. The neoplasm may be composed of cells having a low level of surface expression of Claudin-6. In some cases, low levels of surface expression means that mean number of anti-Claudin-6 antibodies bound per neoplastic cell is less than 20000, such as less than 80000, less than 70000, less than 60000, less than 50000, less than 40000, less than 30000, less than 20000, less than 10000, or less than 5000.

In some aspects, individuals are selected on the basis they have a neoplasm comprising both Claudin-6+ve and Claudin-6-ve cells. The neoplasm may be composed of Claudin-6-ve neoplastic cells, optionally wherein the Claudin-6-ve neoplastic cells are associated with Claudin-6+ve neoplastic or non-neoplastic cells. The neoplasm or neoplastic cells may be all or part of a solid tumour. The solid tumour may be partially or wholly Claudin-6-ve.

In some cases, expression of Claudin-6 in a particular tissue of interest is determined. For example, in a sample of tumor tissue. In some cases, systemic expression of the target is determined. For example, in a sample of circulating fluid such as blood, plasma, serum or lymph.

In some aspects, the individual is selected as suitable for treatment due to the presence or absence of Claudin-6 expression in a sample. In those cases, individuals without Claudin-6 expression may be considered not suitable for treatment.

In other aspects, the level of Claudin-6 expression is used to select a individual as suitable for treatment.

Where the level of expression of Claudin-6 is above a threshold level, the individual is determined to be suitable for treatment.

In some aspects, the presence of Claudin-6 in cells in the sample indicates that the individual is suitable for treatment with an ADC as disclosed herein. In other aspects, the amount of Claudin-6 expression must be above a threshold level to indicate that the individual is suitable for treatment. In some aspects, the observation that Claudin-6 localisation is altered in the sample as compared to a control indicates that the individual is suitable for treatment.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

In another aspect the sample is a biopsy of solid tissue.

The sample may be fresh or archival. For example, archival tissue may be from the first diagnosis of an individual, or a biopsy at a relapse. In certain aspects, the sample is a fresh biopsy.

The terms “subject”, “patient” and “individual” are used interchangeably herein.

In some aspects disclosed herein, an individual has, or is suspected as having, or has been identified as being at risk of, a proliferative disease such as cancer. In some aspects disclosed herein, the individual has already received a diagnosis of such a disease. A list of relevant diseases is provided above in the section “Treated disorders”. Ovarian cancer, non-small cell lung carcinoma (NSCLC), gastric cancer, oesophageal cancer, endometrial cancer and hepatocellular carcinoma (HCC) are conditions of particular interest.

In some cases, the individual has received a diagnosis of a proliferative disease such as cancer, such as one of the disorders listed above. Ovarian cancer, non-small cell lung carcinoma (NSCLC), gastric cancer, oesophageal cancer, endometrial cancer and hepatocellular carcinoma (HCC) are conditions of particular interest.

In some cases, the individual has received a diagnosis of a solid cancer containing Claudin-6+ expressing cells.

The individual may be undergoing, or have undergone, a therapeutic treatment for that cancer. The subject may, or may not, have previously received an anti-Claudin-6 ADC. In some cases the cancer is ovarian cancer, non-small cell lung carcinoma (NSCLC), gastric cancer, oesophageal cancer, endometrial cancer or hepatocellular carcinoma (HCC).

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the ADC alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Also included in the definition of “chemotherapeutic agent” are: (i) anti-hormonal agents that act to regulate or inhibit hormone action on tumors such as anti-estrogens and selective estrogen receptor modulators (SERMs); (ii) aromatase inhibitors that inhibit the enzyme aromatase, which regulates estrogen production in the adrenal glands; (iii) anti-androgens; (iv) protein kinase inhibitors such as MEK inhibitors; (v) lipid kinase inhibitors; (vi) anti-angiogenic agents.

Also included in the definition of “chemotherapeutic agent” are therapeutic antibodies, including bispecific antibodies.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which will typically be by injection, e.g. cutaneous, subcutaneous, or intravenous.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Dosage

It will be appreciated by one of skill in the art that appropriate dosages of the ADC, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of ADC is determined by the expression of Claudin-6 observed in a sample obtained from the subject. Thus, the level or localisation of expression of Claudin-6 in the sample may be indicative that a higher or lower dose of ADC is required. For example, a high expression level of Claudin-6 may indicate that a higher dose of ADC would be suitable. In some cases, a high expression level of Claudin-6 may indicate the need for administration of another agent in addition to the ADC. For example, administration of the ADC in conjunction with a chemotherapeutic agent. A high expression level of Claudin-6 may indicate a more aggressive therapy.

In certain aspects, the dosage level is determined by the expression of Claudin-6 on neoplastic cells in a sample obtained from the subject. For example, when the target neoplasm is composed of, or comprises, neoplastic cells expressing Claudin-6.

In certain aspects, the dosage level is determined by the expression of Claudin-6 on cells associated with the target neoplasm. For example, the target neoplasm may be a solid tumour composed of, or comprising, neoplastic cells that express Claudin-6. For example, the target neoplasm may be a solid tumour composed of, or comprising, neoplastic cells that do not express Claudin-6. The cells expressing Claudin-6 may be neoplastic or non-neoplastic cells associated with the target neoplasm.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

For the ADC, where it is an Exatecan bearing ADC, the dosage amounts described above may apply to the conjugate (including the Exatecan moiety and the linker to the antibody) or to the effective amount of Exatecan compound provided, for example the amount of compound that is releasable after cleavage of the linker.

### Specific Embodiments

Embodiment 1—An antibody drug conjugate of formula (I): Ab-L-Dp, wherein:
Ab is an antibody that binds to Claudin-6, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;
L-Dp is a drug linker conjugated to the Ab, such as via one or more cysteine residues, wherein L typically comprises a polyglycine moiety, Glyₙ wherein n is from 5 to 8, and D is an Exatecan, wherein the drug linker can be cleaved under cellular conditions to release an Exatecan with the following formula (II):

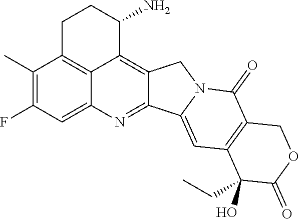 [SMILES: CC[C@@]1(O)C(=O)OCc2c1cc1n(c2=O)Cc2c-1nc1cc(F)c(C)c3c1c2[C@@H](N)CC3]

and wherein p is the number of drug units per antibody and is from 1 to 8, such as 1 to 6.

Embodiment 2—A conjugate according to embodiment 1 wherein the antibody has a VH domain as shown in SEQ ID NO: 1.

Embodiment 3—A conjugate according to embodiment 1 or embodiment 2 wherein the antibody has a VL domain as shown in SEQ ID NO: 2.

Embodiment 4—A conjugate according to any one of embodiments 1 to 3 wherein Ab has a mutation in one, two or three hinge region cysteines (such as one mutation to leave three sites available per heavy/light chain, six in total).

Embodiment 5—A conjugate according to embodiment 4 wherein Ab has a mutation in Cysteine 226 (EU numbering) of the heavy chain, such as a Cys to Valine substitution.

Embodiment 6—A conjugate according to any one of the preceding embodiments wherein Ab comprises the following heavy chain mutations: a Leucine 234 to Alanine substitution, a Leucine 235 to Alanine substitution and a Proline 329 to Alanine substitution (EU numbering).

Embodiment 7—A conjugate according to any one of the preceding embodiments wherein Ab has an engineered cysteine outside the hinge region, such as in the light chain, such as a V205C mutation.

Embodiment 8—A conjugate according to any one of the preceding embodiments wherein the linker comprises -G^{LL}-X-Glyₙ-A- wherein G^{LL} is for connection of the linker to a cysteine residue on the antibody and is as defined herein; X is an optional spacer portion as defined herein; n is from 5 to 8; and A comprises a moiety cleavable under cellular conditions and optionally a self-immolative moiety.

Embodiment 9—A conjugate according to any one of the preceding embodiments wherein the linker comprises a cathepsin cleavable sequence e.g. Val-Ala or Val-Cit.

Embodiment 10—A conjugate according to any one of the preceding embodiments wherein the linker comprises a self-immolative moiety operably linked to the Exatecan, such as para-aminobenzylcarbamate.

Embodiment 11—A conjugate according to any one of the preceding embodiments where L-Dp is:

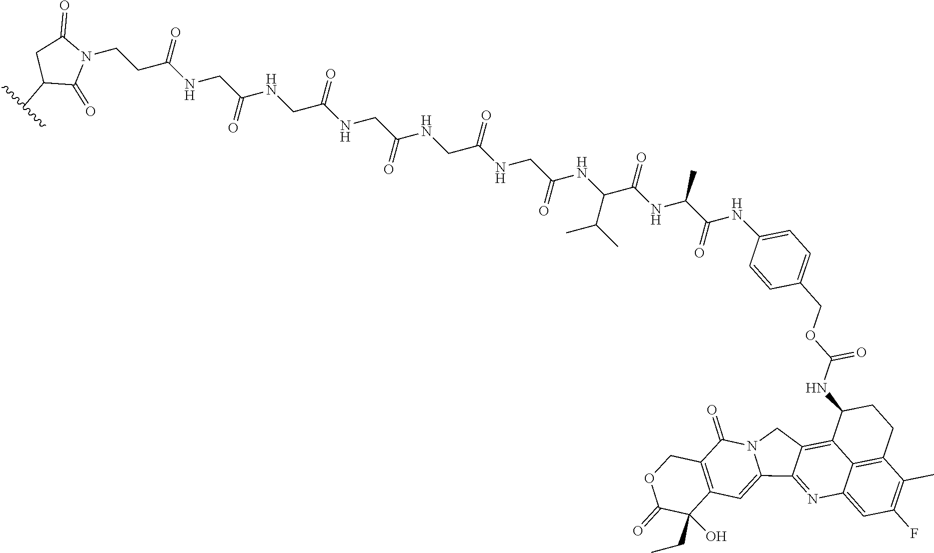 [SMILES: *C1CC(=O)N(CCC(=O)NCC(=O)NCC(=O)NCC(=O)NCC(=O)NCC(=O)NC(C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

Embodiment 12—A composition comprising a mixture of antibody drug conjugates according to any one of embodiments 1 to 11 wherein the average drug to antibody ratio is from 1 to 2 or 3 to 6.

Embodiment 13—A pharmaceutical composition comprising a conjugate or composition according to any one of the previous embodiments together with one or more pharmaceutically acceptable carriers or diluents, such as a pharmaceutical composition in liquid or lyophilized form.

Embodiment 14—An antibody drug conjugate according to any one of embodiments 1 to 11 or a composition according to embodiment 12 or embodiment 13 for use in treating a proliferative disease, for example a disease characterized by over-expression of Claudin-6.

Embodiment 15—An antibody drug conjugate or composition for use in treating in an individual a cancer selected from ovarian cancer, lung cancer (such as non-small cell lung cancer), oesophageal cancer, liver cancer such as hepatocellular carcinoma (HCC), endometrial cancer, gastric cancer, testicular cancer, pancreatic cancer, bronchial cancer, breast cancer, ear nose and throat (ENT) cancer, colon cancer, head and neck cancer (such as head and neck squamous cell cancer (HNSCC)), gallbladder cancer, gliomas (such as adult high grade gliomas) and atypical teratoid rhabdoid tumors, and their metastases (e.g. gastric cancer metastasis such as Krukenberg tumor, peritoneal metastasis or lymph node metastasis).

Embodiment 16—A method of treating an individual suffering from a proliferative disease, for example a disease characterized by over-expression of Claudin-6, which method comprises administering to the patient an antibody drug conjugate according to any one of the embodiments 1 to 11 or a composition according to embodiment 12 or embodiment 13.

Embodiment 17—A method of treating an individual patient suffering cancer selected from ovarian cancer, lung cancer (such as non-small cell lung cancer), oesophageal cancer, liver cancer such as hepatocellular carcinoma (HCC), endometrial cancer, gastric cancer, testicular cancer, pancreatic cancer, bronchial cancer, breast cancer, ear nose and throat (ENT) cancer, colon cancer, head and neck cancer (such as head and neck squamous cell cancer (HNSCC)), gallbladder cancer, gliomas (such as adult high grade gliomas) and atypical teratoid rhabdoid tumors, and their metastases (e.g. gastric cancer metastasis such as Krukenberg tumor, peritoneal metastasis or lymph node metastasis). which method comprises administering to the individual an antibody drug conjugate according to any one of embodiments 1 to 11 or a composition according to embodiment 12 or embodiment 13.

### Sequence Listing Part of the Description

| [GB01 VH, CDR underline] | |
|---|---|
| SEQ ID NO. 1 | |
| 60 | QVQLQESGPG LVKPSETLSL TCTVSGYSIT SGYYWNWIRQ PPGKGLEWMG YISYSGSNNY |
| 116 | NPSLKNRVTI SRDTSKNQFS LKLSSVTAAD TAVYYCARGG YYFDYWGQGT LVTVSS |
| [GB01 VL, CDR underline] | |
| SEQ ID NO. 2 | |
| 60 | DIVMTQSPDS LAVSLGERAT INCKSSQSLL NTNNQKNYLA WYQQKPGQPP KLLIYFASTR |
| 113 | ESGVPDRFSG SGSGTDFTLT ISSLQAEDVA VYFCQQHYNA PRTFGGGTKV EIK |

| [GB01 VH, Kabat CDR1] | |
|---|---|
| SEQ ID NO. 3 | |
| SGYYWN | |
| [GB01 VH, Kabat CDR2] | |
| SEQ ID NO. 4 | |
| YISYSGSNNY NPSLKN | |
| [GB01 VH, Kabat CDR3] | |
| SEQ ID NO. 5 | |
| GGYYFDY | |
| [GB01 VL, Kabat CDR1] | |
| SEQ ID NO. 6 | |
| KSSQSLLNTN NQKNYLA | |
| [GB01 VL, Kabat CDR2] | |
| SEQ ID NO. 7 | |
| FASTRES | |
| [GB01 VL, Kabat CDR3] | |
| SEQ ID NO. 8 | |
| QQHYNAPRT | |
| [GB01 Full Length Heavy Chain] | |
| SEQ ID NO. 9 | |
| QVQLQESGPG LVKPSETLSL TCTVSGYSIT SGYYWNWIRQ PPGKGLEWMG YISYSGSNNY | 60 |
| NPSLKNRVTI SRDTSKNQFS LKLSSVTAAD TAVYYCARGG YYFDYWGQGT LVTVSSASTK | 120 |
| GPSVFPLAPS SKSTSGGTAA LGCLVKDYFP EPVTVSWNSG ALTSGVHTFP AVLQSSGLYS | 180 |
| LSSVVTVPSS SLGTQTYICN VNHKPSNTKV DKKVEPKSCD KTHTCPPCPA PELLGGPSVF | 240 |
| LFPPKPKDTL MISRTPEVTC VVVDVSHEDP EVKFNWYVDG VEVHNAKTKP REEQYNSTYR | 300 |
| VVSVLTVLHQ DWLNGKEYKC KVSNKALPAP IEKTISKAKG QPREPQVYTL PPSRDELTKN | 360 |
| QVSLTCLVKG FYPSDIAVEW ESNGQPENNY KTTPPVLDSD GSFFLYSKLT VDKSRWQQGN | 420 |
| VFSCSVMHEA LHNHYTQKSL SLSPGK | 446 |
| [GB01 Full Length heavy chin with hinge Cys and PALALA substitutions in bold] | |
| SEQ ID NO. 10 | |
| QVQLQESGPG LVKPSETLSL TCTVSGYSIT SGYYWNWIRQ PPGKGLEWMG YISYSGSNNY | 60 |
| NPSLKNRVTI SRDTSKNQFS LKLSSVTAAD TAVYYCARGG YYFDYWGQGT LVTVSSASTK | 120 |
| GPSVFPLAPS SKSTSGGTAA LGCLVKDYFP EPVTVSWNSG ALTSGVHTFP AVLQSSGLYS | 180 |
| LSSVVTVPSS SLGTQTYICN VNHKPSNTKV DKKVEPKSCD KTHTVPPCPA PEAAGGPSVF | 240 |
| LFPPKPKDTL MISRTPEVTC VVVDVSHEDP EVKFNWYVDG VEVHNAKTKP REEQYNSTYR | 300 |
| VVSVLTVLHQ DWLNGKEYKC KVSNKALAAP IEKTISKAKG QPREPQVYTL PPSRDELTKN | 360 |
| QVSLTCLVKG FYPSDIAVEW ESNGQPENNY KTTPPVLDSD GSFFLYSKLT VDKSRWQQGN | 420 |
| VFSCSVMHEA LHNHYTQKSL SLSPGK | 446 |
| [GB01 Full Length Light Chain] | |
| SEQ ID NO. 11 | |
| DIVMTQSPDS LAVSLGERAT INCKSSQSLL NTNNQKNYLA WYQQKPGQPP KLLIYFASTR | 60 |
| ESGVPDRFSG SGSGTDFTLT ISSLQAEDVA VYFCQQHYNA PRTFGGGTKV EIKRTVAAPS | 120 |
| VFIFPPSDEQ LKSGTASVVC LLNNFYPREA KVQWKVDNAL QSGNSQESVT EQDSKDSTYS | 180 |
| LSSTLTLSKA DYEKHKVYAC EVTHQGLSSP VTKSFNRGEC | 220 |
| [GB01 Full Length Light Chain-V205C-5T] | |
| SEQ ID NO. 12 | |
| DIVMTQSPDS LAVSLGERAT INCKSSQSLL NTNNQKNYLA WYQQKPGQPP KLLIYFASTR | 60 |
| ESGVPDRFSG SGSGTDFTLT ISSLQAEDVA VYFCQQHYNA PRTFGGGTKV EIKRTVAAPS | 120 |
| VFIFPPSDEQ LKSGTASVVC LLNNFYPREA KVQWKVDNAL QSGNSQESVT EQDSKDSTYS | 180 |
| LSSTLTLSKA DYEKHKVYAC EVTHQGLSSP CTKSFNRGEC | 220 |
| [GB03 Full Length Heavy Chain] | |
| SEQ ID NO. 13 | |
| EVQLVESGGG LVQPGGSLRL SCAASGFTFS IYGMSWVRQA PGKGLEWVAT ISRDASYTYF | 60 |
| PDSVKGRFTI SRDNAKNSLY LQMNSLRAED TAVYYCVRLG DNDRGYAMDY WGQGTLVTVS | 120 |
| SASTKGPSVF PLAPSSKSTS GGTAALGCLV KDYFPEPVTV SWNSGALTSG VHTFPAVLQS | 180 |
| SGLYSLSSVV TVPSSSLGTQ TYICNVNHKP SNTKVDKKVE PKSCDKTHTC PPCPAPELLG | 240 |
| GPSVFLFPPK PKDTLMISRT PEVTCVVVDV SHEDPEVKFN WYVDGVEVHN AKTKPREEQY | 300 |
| NSTYRVVSVL TVLHQDWLNG KEYKCKVSNK ALPAPIEKTI SKAKGQPREP QVYTLPPSRD | 360 |
| ELTKNQVSLT CLVKGFYPSD IAVEWESNGQ PENNYKTTPP VLDSDGSFFL YSKLTVDKSR | 420 |
| WQQGNVFSCS VMHEALHNHY TQKSLSLSPG K | 451 |
| [GB03 Full Length Light Chain] | |
| SEQ ID NO. 14 | |
| DVVMTQSPLS LPVTLGQPAS ISCRSSQSVV HVNANTYLEW YQQRPGQSPR LLIYKVSNRF | 60 |
| PGVPDRFSGS GSGTDFTLKI SRVEAEDVGV YYCFQGSHVP RTFGGGTKVE IKRTVAAPSV | 120 |
| FIFPPSDEQL KSGTASVVCL LNNFYPREAK VQWKVDNALQ SGNSQESVTE QDSKDSTYSL | 180 |
| SSTLTLSKAD YEKHKVYACE VTHQGLSSPV TKSFNRGEC | 219 |

Some aspects and embodiments of the disclosure are described below in more detail with reference to the following examples, which are illustrative only and non-limiting.

### EXAMPLES

### Preparation of Drug Linkers

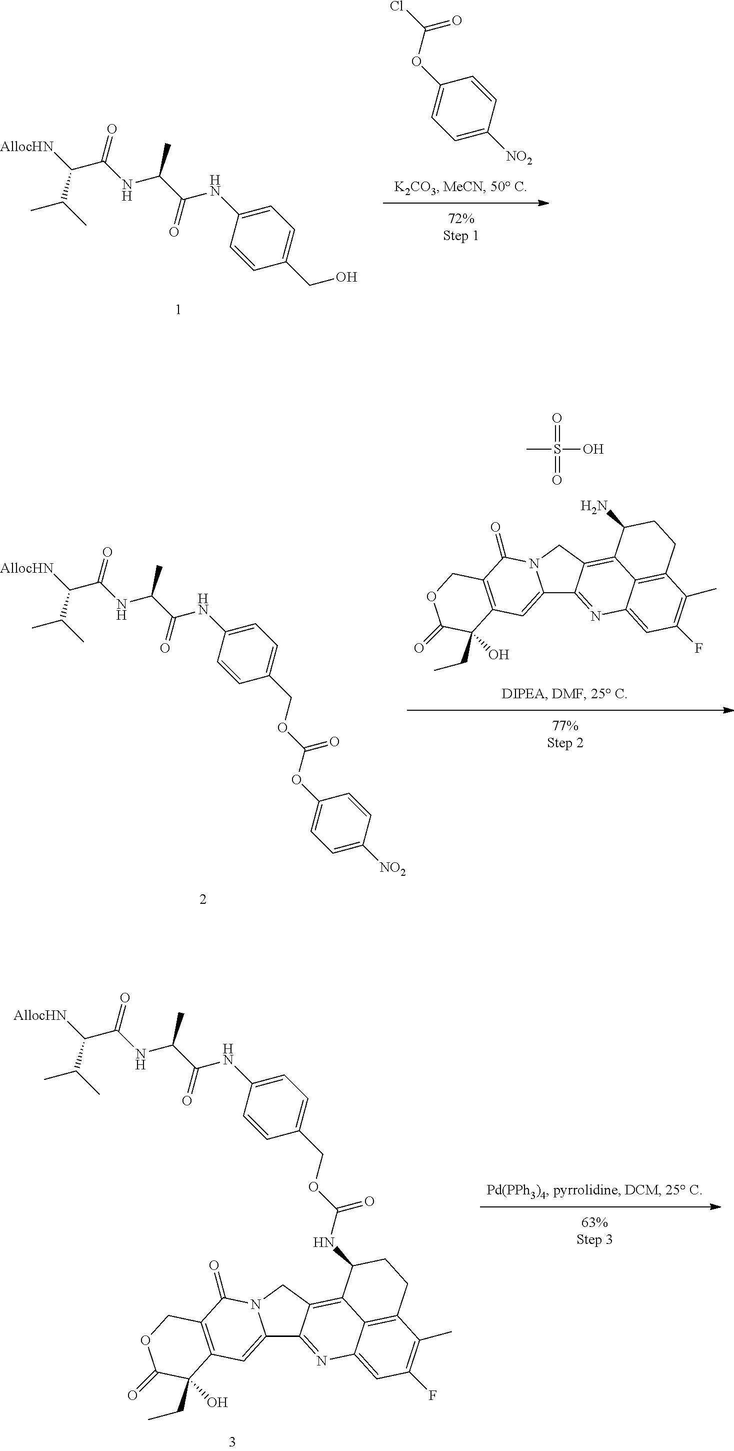

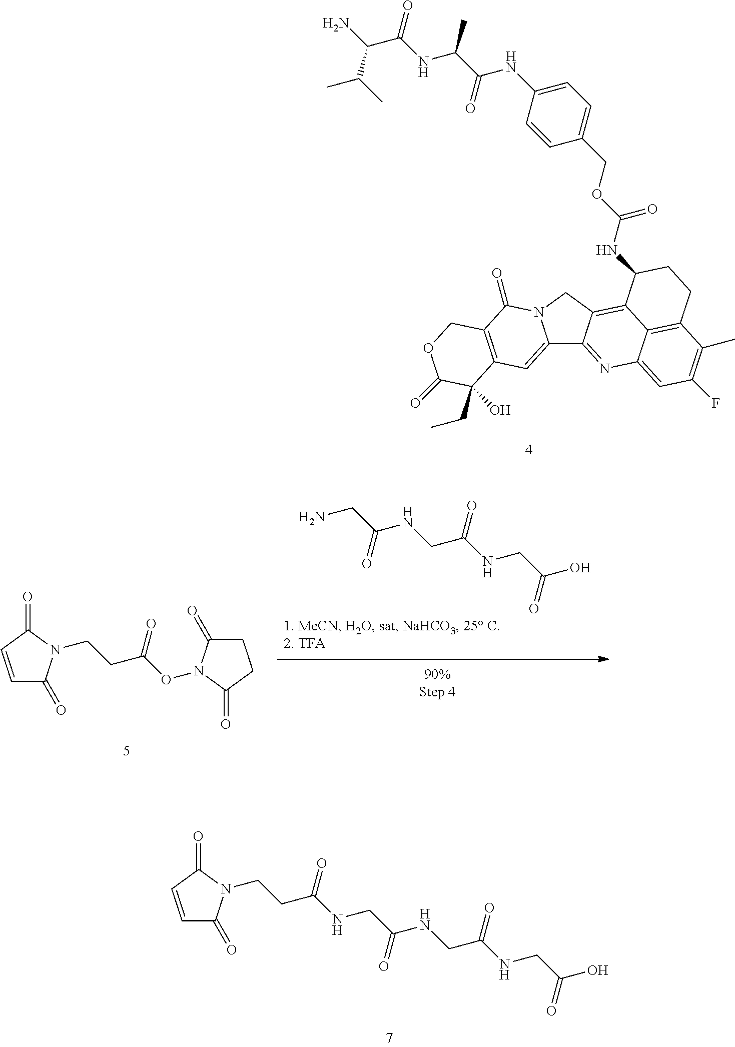 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](N)C(C)C)cc1)CC3 ; O=C(CCN1C(=O)C=CC1=O)ON1C(=O)CCC1=O ; NCC(=O)NCC(=O)NCC(=O)O ; O=C(O)CNC(=O)CNC(=O)CNC(=O)CCN1C(=O)C=CC1=O]

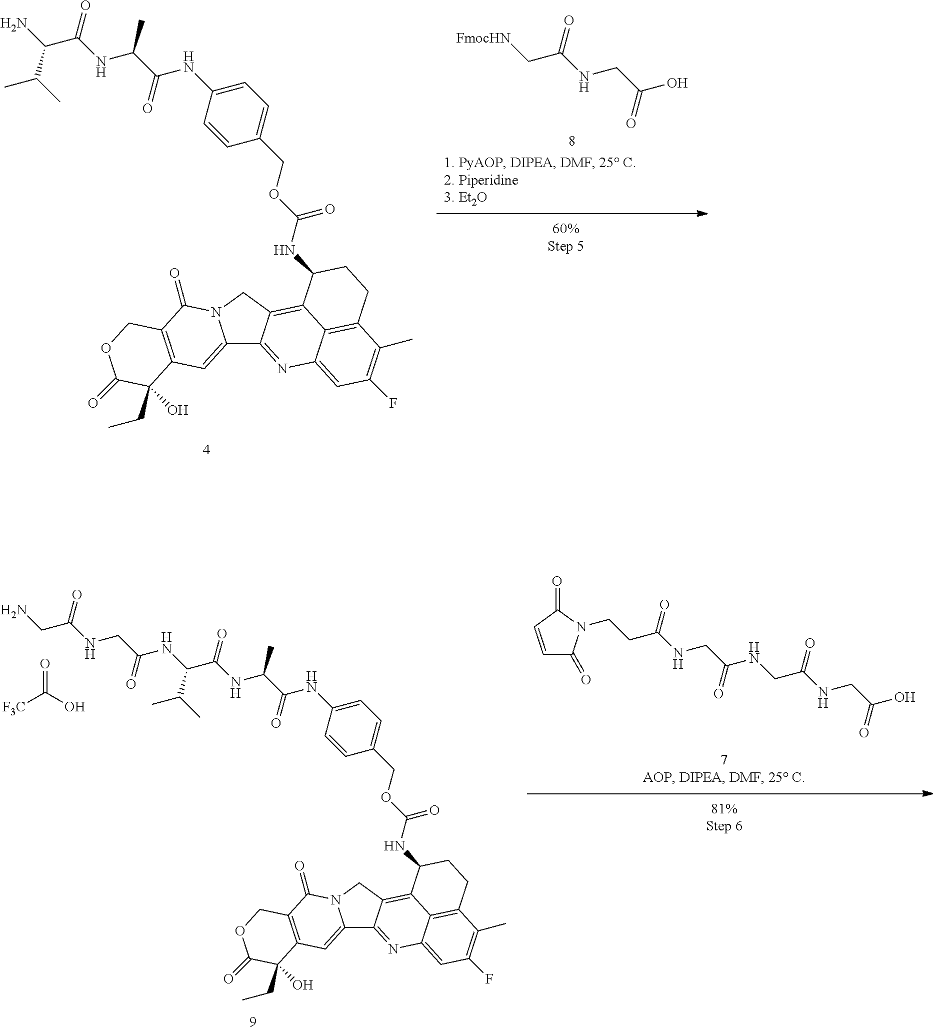

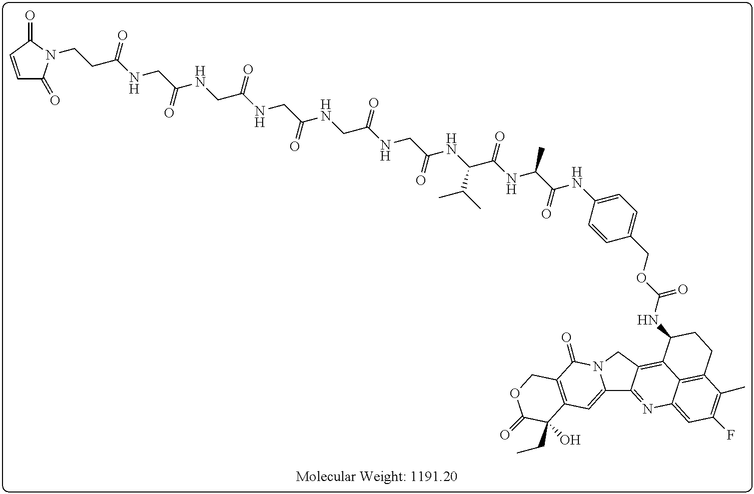 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](C)NC(=O)[C@@H](NC(=O)CNC(=O)CNC(=O)CNC(=O)CNC(=O)CNC(=O)CCN2C(=O)C=CC2=O)C(C)C)cc1)CC3]

### Step 1:

Alloc-Val-Ala-PABA (1) (29 g, 1 eq) was dissolved in MeCN (10 V). K₂CO₃ (2 eq) was added. The reaction mixture was heated to 50° C. A solution of 4-nitrophenyl carbonochloridate in MeCN (2 eq in 5 V) was added. The reaction mixture was stirred at 50° C. for 6 h. On completion, the reaction mixture was filtered.

The filtrate was concentrated in vacuo to give the crude residue. The latter was purified by flash column chromatography (silica) eluting with EtOAc/DCM (0->20%) to afford compound 2 (30 g, 72%).

### Step 2:

Exatecan mesylate (24 g, 1 eq) was dissolved in DMF (10 V). DIPEA (3 eq) was added. Compound 2 (1.25 eq) was added and the reaction mixture was stirred at 25° C. for 16 h.

On completion, water (30 V) was added dropwise to the reaction mixture. The latter was stirred at 25° C. for 30 min. The reaction mixture was filtered and the cake was washed with water (5 V), collected and thoroughly dried to afford compound 3 (29 g, 77%).

### Step 3:

Compound 3 (30 g, 1 eq) was dissolved in DCM (10 V). Pd(PPh₃)₄ (0.02 eq) and pyrrolidine (1.5 eq) were added. The reaction mixture was stirred at 25° C. for 2 h. On completion, the reaction mixture was concentrated in vacuo to give the crude residue. The latter was purified by flash column chromatography (silica) eluting with MeOH/DCM (0->8%) to afford compound 4 (17 g, 63%).

### Step 4:

To a solution of compound 5 (5.32 g, 20 mmol) and compound 6 (3.8 g, 20 mmol) in acetonitrile/water (1/2, v/v, 100 mL) was added sat. NaHCO₃ solution (40 mL).

The reaction was stirred at room temperature for 2 h. Then TFA (2.8 mL) was added and the stirring continued for an additional 10 min. The mixture was purified directly by preparative RP-HPLC to afford compound 7 as a white solid (6.1 g, 90%).

### Step 5:

To a solution of compound 4 (3.08 g, 4.08 mmol) and compound 8 (1.44 g, 4.08 mmol) in anhydrous DMF (50 mL) was added PyAOP (2.13 g, 4.08 mmol) and DIPEA

(2.1 mL, 12.08 mmol). The reaction was stirred at room temperature for 10 min. Then piperidine (2.0 mL) was added and the stirring continued for an additional 10 min.

The mixture was added to diethyl ether (500 mL) while stirring. The suspension was centrifugated and the precipitate was collected by decantation. Then the precipitate was dissolved in 40 mL of DMF and purified by preparative RP-HPLC to give compound 9 as a yellow solid, TFA salt (2.4 g, 60%).

### Step 6:

To a solution of compound 9 (TFA salt, 814 mg, 0.83 mmol) and compound 7 (282 mg, 0.83 mmol) in anhydrous DMF (20 mL) was added AOP (367 mg, 0.83 mmol) and DIPEA (0.57 mL, 3.28 mmol). The reaction was stirred at room temperature for 10 min. Then the mixture was purified by preparative RP-HPLC to afford compound DL-A as a yellow solid (510 mg, 81%).

### Preparative RP-HPLC Method:

Column: Phenomenex Gemini NX 5m, C18, 110 Å, 150×50 mm
Instrument: Shimadzu LC with CTC IFC
Mobile phase: A) water (0.1% TFA), B) acetonitrile
Gradient: 10-65% B over 20 min, flow 50 mL/min

### Analytical HPLC:

Column: Phenomenex Gemini NX 5m, C18, 110 Å, 150×4.6 mm
Instrument: Shimadzu LC-2030
Mobile phase: A) water (0.05% TFA), B) acetonitrile (0.05% TFA)
Gradient: 20-70% B over 15 min, flow 1.0 mL/min

All the desired HPLC fractions were pooled and lyophilised on a Virtis Freezemobile 35EL.

### Synthesis of ADCs

Different ADCs were constructed linked to antibodies GB01 or GB03, which are humanised anti-Claudin-6 antibodies. GB01 has been engineered to remove a hinge region cysteine (C226V of the heavy chain (EU numbering), to control the drug to antibody ratio (DAR). A further modification has been made on the heavy chain to reduce Fc receptor binding (L234A/L235A/P329A). The sequences of the GB01 heavy and light chains are shown in SEQ ID NOs: 10 and 12, respectively.

ADC-1 has Exatecan as the warhead with a maleimide-Gly5-Val-Ala-PABA linker (DL-A) (SEQ ID NO: 21) conjugated to antibody GB01 via hinge region cysteine resides essentially as described in Zammarchi et al., 2016. Mol Cancer Ther 15: 2709. In brief, antibody was buffer exchanged into a histidine buffer at pH 6 using tangential flow filtration, pH was adjusted to 7.5 using a TRIS/EDTA pH 8.5 buffer, and the solution was reduced with Tris (2-carboxyethyl) phosphine reductant. Dimethylacetamide and drug-linker (threefold excess relative to antibody) were added to the solution. The conjugation reaction was incubated, then quenched with threefold molar excess of N-acetyl cysteine and incubated again. The pH was then decreased to 6.0 using histidine hydrochloride solution and the generated ADC1 was purified by tangential flow filtration, filtered, and stored at −70° C. Final yield was estimated by ultraviolet-visible spectrophotometry based on starting antibody. Synthesis of the drug linker DL-A is described above.

ADC-2 is the same as ADC1 but conjugated to antibody GB03.

ADC-3 is SG3249 (a pyrrolobenzodiazepine warhead, SG3199) as the toxin, with a maleimide-PEG8-Val-Ala-PABA linker) conjugated to antibody GB03 essentially as for ADC1.

ADC-4 is essentially the same as ADC-1 but conjugated to antibody B12, a human IgG1 non-binding antibody, as an isotype control.

ADC-5 is a comparator ADC using a different anti-Claudin 6 antibody conjugated to an MMAE warhead with a ValCit-PABA linker (McDermott et al., 2023, Clin Cancer Res. 29(11): 2131-2143).

ADC-6 is the same as ADC-5 but conjugated to antibody B12, a human IgG1 non-binding antibody, as an isotype control.

### Aggregation Studies

2 mg of monoclonal antibody GB01 at 8.87 mg/ml was pH adjusted with 5% 0.5 M Tris, 0.025 M EDTA, pH 8.5 and reduced with 2.3 mol. eq. TCEP and then conjugated with 6 mol. eq. drug linker. After buffer exchange into 30 mM Histidine, 175 mM Sucrose pH 6.0/PBS, the drug to antibody ratio (DAR) was by HIC/LC-MS and % Monomer by size exclusion chromatography (SEC). Percentage monomer values of 95% or greater are assessed as low aggregation.

### Example 1—Aggregation Studies with Exatecan Linkers

Previous work that we have conducted using PEG-based linkers and an Exatecan warhead showed unfavourable aggregation properties. Therefore we sought to identify an alternative linker design. A new linker was designed using multiple glycine residues instead of PEG and this led to an improvement in levels of aggregation (data not shown). This glycine linker concept was then tested in new constructs based on a maleimide conjugation group and a Val-Ala-PABC cleavable portion linked to Exatecan.

A maleimide-Gly3-Val-Ala-PABC-Exatecan (SEQ ID NO: 22) construct was tested against a maleimide-Gly5-Val-Ala-PABC-Exatecan construct (DL-A, the synthesis of which is described above—the Gly3 variant differs only with respect to the number of Gly residues) (SEQ ID NO: 21). Log P values (a measure of hydrophobicity) were estimated using CDD Vault software (Collaborative Drug Discovery Inc.) based on the method described in Gedeck et al., 2017, J. Chem. Inf. Model. 57, 8, 1847-1858. Model training information using publicly available datasets is available from CDD. The results are shown in Table 1.

A correlation study was also carried out using Reverse-Phase HPLC (RP-HPLC). The two constructs were injected in RP-HPLC and retention times (Rt) correlated with hydrophobicity—i.e., high Rt was associated to hydrophobic constructs whereas low Rt to hydrophilic ones. The results are shown in Table 1.

Aggregation levels were assessed during the conjugation to a test antibody. Conjugation was performed as described above for ADC1.

During conjugation, we are ideally targeting a monomer content or percentage monomer of 95% or above. Low percentage monomer values usually translate to a higher degree of aggregation. One could therefore agree that percentage monomer values of 95% or higher mean very low levels of aggregation or none, whereas values below this threshold often translate to higher levels of aggregation.

### Results

TABLE 1

| | | | Aggregation |
|---|---|---|---|
| Construct ID | cLogP | Rt (min) | (% monomer) |
| Mal-Gly5-VA-PABC-Exatecan | −0.81 | 9.76 | None (98%) |
| (PL2202) | | | |
| Mal-Gly3-VA-PABC-Exatecan | 0.59 | 9.98 | Moderate (87%) |
| (PL2208) | | | |

### Discussion

These results clearly show the hydrophilicity-enhancing properties in a drug linker construct of a novel pentaglycine spacer/linker. The incorporation of this Gly5 motif allowed constructs to exhibit increased hydrophilicity and lower aggregation levels for optimal conjugation processes. Gly5-based constructs showed no aggregation versus moderate aggregation for a Gly3-based version.

### Example 2—In Vivo Efficacy of Anti-Claudin-6 Antibody Conjugated to Different Payloads in the PA1 Ovarian Xenograft Model

Female athymic NCr nu/nu nude mice (Crl:NU(NCr)-Foxn1 nu, Charles River) were six to eight weeks old with a body weight (BW) range of 18.3 to 22.9 g on Day 1 of the study. Each mouse was injected subcutaneously (s.c.) in the right flank with 1×10⁷ PA-1 cells in 50% matrigel. Tumor were measured in two dimensions using calipers, and volume was calculated using the formula:

$$Tumor Volume ( {mm}^{3} ) = {w}^{2} \times l / 2$$

where w=width and l=length, in mm, of the tumor. Tumor weight may be estimated with the assumption that 1 mg is equivalent to 1 mm³ of tumor volume.

Eight days later, designated as Day 1 of the study, mice were sorted into treatment groups (n=10 per group) with group mean tumor volumes of about 140 mm³. On Day 1 of the study, drugs were administered intravenously (i.v.) in a single injection (qd×1) via tail vein injection. The dosing volume was 0.2 mL per 20 grams of body weight (10 mL/kg) and was scaled to the body weight of each individual animal. Tumors were measured using calipers twice per week, and each animal was euthanized when its tumor reached the endpoint volume of 3000 mm³ or at the end of the study (Day 35), whichever came first.

### Results

TABLE 2
In vivo anti-tumour activity based on Mean Tumor
Volume mm³ at days 1 and 42 (mean of n = 10)

| Response summary | Day 1 | Day 35 | % change |
|---|---|---|---|
| Vehicle | 140 | 2597 | +1754% |
| ADC-1 (12 mg/kg) (GB01 Exatecan) | 140 | 691 | +390% |
| ADC-2 (12 mg/kg) (GB03 Exatecan) | 140 | 1919 | +1267% |
| ADC-3 (0.5 mg/kg) (GB03 PBD) | 140 | 1051 | +650% |

### Conclusions

The Exatecan ADC using the GB01 antibody was more efficacious than the same Exatecan ADC using the GB03 antibody as well as the PBD ADC using the GB03 antibody.

### Example 3—In Vivo Efficacy of Anti-Claudin-6 Antibody Conjugated to Exatecan Payload Compared to a Benchmark MMAE ADC in OVCAR3 Xenograft Model

Female CB.17 SCID mice (Charles River) were ten weeks old with a body weight (BW) range of 17.0 to 22.6 g on Day 0 of the study. Each mouse was anesthetized with isoflurane for subcutaneous surgical implantation of ˜4 mm³ of OVCAR-3 fragment in the right flank. Tumors were measured as per Example 2.

Fifteen days later, designated as Day 0 of the study, mice were sorted into treatment groups (n=8 per group) with individual tumor volumes ranging from 75 to 196 mm3 and group mean tumor volumes of 123 mm³. On Day 0 of the study, ADCs 1 and 4 were administrated intravenously (i.v.) in a single injection (qd×1); ADCs 5 and 6 were administered i.v. once a week for three weeks. The dosing volume was 0.2 mL per 20 grams of body weight (10 mL/kg) and was scaled to the body weight of each individual animal. Tumors were measured using calipers twice per week, and each animal was euthanized when its tumor reached the endpoint volume of 1000 mm³ or at the end of the study (Day 42), whichever came first.

### Results

TABLE 3
In vivo anti-tumour activity

| Response summary | PR | CR | TFS |
|---|---|---|---|
| Vehicle | 0 | 0 | 0 |
| ADC-1 (0.5 mg/kg) - (GB01 Exatecan) | 0 | 0 | 0 |
| ADC-1 (1 mg/kg) - (GB01 Exatecan) | 3 | 2 | 1 |
| ADC-1 (2 mg/kg) - (GB01 Exatecan) | 0 | 6 | 5 |
| ADC-4 (2 mg/kg) - (B12 Exatecan) | 0 | 0 | 0 |
| ADC-5 (2.5 mg/kg) - (CLDN6-MMAE) | 1 | 0 | 0 |
| ADC-5 (5 mg/kg) - (CLDN6-MMAE) | 0 | 0 | 0 |
| ADC-6 (2.5 mg/kg) - (B12 MMAE) | 0 | 0 | 0 |
| ADC-6 (5 mg/kg) - (B12 MMAE) | 0 | 0 | 0 |

PR = partial responder.
CR = complete responder.
TFS = tumour-free survivor

In a PR response, the tumor volume was 50% or less of its Day 0 volume for three consecutive measurements during the course of the study, and equal to or greater than 13.5 mm³ for one or more of these three measurements. In a CR response, the tumor volume was less than 13.5 mm³ for three consecutive measurements during the course of the study. An animal with a CR response at the termination of a study was additionally classified as a tumor-free survivor (TFS).

TABLE 4
In vivo anti-tumour activity based on Mean Tumor
Volume mm³ at days 0 and 42 (mean of n = 8)

| Response summary | Day 0 | Day 35 | % change |
|---|---|---|---|
| Vehicle | 123.25 | 1039 | +743% |
| ADC-1 (0.5 mg/kg) (GB01 Exatecan) | 123.25 | 1010.86 | +720.17% |
| ADC-1 (1 mg/kg) (GB01 Exatecan) | 123.25 | 105.13 | −14.70% |
| ADC-1 (2 mg/kg) (GB01 Exatecan) | 123.25 | 239.63 | +94.43% |
| ADC-4 (2 mg/kg) (B12 Exatecan) | 123.25 | 1045.88 | +748.58% |
| ADC-5 (2.5 mg/kg) (CLDN6-MMAE) | 123.25 | 1055.63 | +756.49% |
| ADC-5 (5 mg/kg) (CLDN6-MMAE) | 123.25 | 1055.63 | +756.49% |
| ADC-6 (2.5 mg/kg) (B12 MMAE) | 123.25 | 1038.25 | +742.39% |
| ADC-6 (5 mg/kg) (B12 MMAE) | 122.86 | 956.29 | +678.36% |

### Conclusions

The GB01 Exatecan ADC at 1 and 2 mg/kg was more efficacious than the benchmark ADC with an MMAE payload.

### Example 4—In Vivo Efficacy of Anti-Claudin-6 Antibody Conjugated to Exatecan Payload Compared to Benchmark ADC in OVCAR3 Xenograft Model

Female CB.17 SCID mice (Charles River) were ten weeks old with a body weight (BW) range of 16.7 to 20.8 g on Day 0 of the study. Each mouse was injected subcutaneously (s.c.) in the right flank with 1×10⁷ OV-90 cells in 50% matrigel. Tumors were measured as per Example 2.

Fifteen days later, designated as Day 0 of the study, mice were sorted into treatment groups (n=10 per group) with individual tumor volumes ranging from 75 to 102 mm³ and group mean tumor volumes of 123 and 125 mm³. On Day 0 of the study, ADCs 1 and 2 were administrated intravenously (i.v.) in a single injection (qd×1); ADCs 5 and 6 were administered i.v. once a week for three weeks. The dosing volume was 0.2 mL per 20 grams of body weight (10 mL/kg) and was scaled to the body weight of each individual animal. Tumors were measured using calipers twice per week, and each animal was euthanized when its tumor reached the endpoint volume of 2000 mm³ or at the end of the study (Day 42), whichever came first.

### Results

TABLE 5
In vivo anti-tumour activity

| Response summary | PR | CR | TFS |
|---|---|---|---|
| Vehicle | 0 | 0 | 0 |
| ADC-1 (12 mg/kg) (GB01 Exatecan) | 4 | 5 | 0 |
| ADC-2 (12 mg/kg) (B12 Exatecan) | 0 | 0 | 0 |
| ADC-5 (2.5 mg/kg) (CLDN6-MMAE) | 0 | 1 | 1 |
| ADC-4 (2.5 mg/kg) (B12 MMAE) | 0 | 0 | 0 |

TABLE 6
In vivo anti-tumour activity based on Mean Tumor
Volume mm³ at days 0 and 42 (mean of n = 10)

| Response summary | Day 0 | Day 35 | % change |
|---|---|---|---|
| Vehicle | 122.90 | 1957.40 | +1492.68% |
| ADC-1 (12 mg/kg) (GB01 Exatecan) | 124.70 | 228.0 | +82.84% |
| ADC-4 (12 mg/kg) (B12 Exatecan) | 124.70 | 807.30 | +547.39% |
| ADC-5 (2.5 mg/kg) (CLDN6-MMAE) | 124.70 | 851.20 | +582.60% |
| ADC-6 (2.5 mg/kg) (B12 MMAE) | 124.70 | 1627.50 | +1205.13% |

### Conclusions

The GB01 Exatecan ADC was more efficacious than the benchmark ADC with an MMAE payload.

### Example 5—In Vivo Efficacy of Anti-Claudin-6 Antibody Conjugated to Exatecan in Lung Patient-Derived Xenograft (PDX) Models

Female BALB/c Nude mice or NOD/SCID mice (GemPharmatech Co., Ltd) were six to nine weeks old with a body weight (BW) range of 20.1 to 28.2 g on Day 0 of the study. Each mouse was injected subcutaneously (s.c.) in the right flank with 2-3 mm in diameter of tumour fragments. Tumors were measured as per Example 2.

Nineteen to fifty-one days later, designated as Day 0 of the study, mice were sorted into treatment groups (n=3-5 per group) with individual tumor volumes ranging from 118.76 to 189.14 mm³ and group mean tumor volumes of 132.67 to 163.55 mm³. On Day 0 of the study, drugs were administered intravenously (i.v.) in a single injection (qd×1) via tail vein injection. The dosing volume was 0.2 mL per 20 grams of body weight (10 mL/kg) and was scaled to the body weight of each individual animal. Tumors were measured using calipers twice per week, and each animal was euthanized when its tumor reached the endpoint volume of 3000 mm³ or at the end of the study (Day 42), whichever came first.

### Results

TABLE 7
In vivo anti-tumour activity in LU2049 model

| Response summary | PR | CR | TFS |
|---|---|---|---|
| Vehicle | 0 | 0 | 0 |
| ADC-1 (10 mg/kg) (GB01 Exatecan) | 0 | 4 | 4 |
| ADC-4 (10 mg/kg) (B12 Exatecan) | 0 | 0 | 0 |

TABLE 8
In vivo anti-tumour activity in LU1144 model

| Response summary | PR | CR | TFS |
|---|---|---|---|
| Vehicle | 0 | 0 | 0 |
| ADC-1 (10 mg/kg) (GB01 Exatecan) | 1 | 0 | 0 |
| ADC-4 (10 mg/kg) (B12 Exatecan) | 0 | 0 | 0 |

TABLE 9
In vivo anti-tumour activity in LU11836 model

| Response summary | PR | CR | TFS |
|---|---|---|---|
| Vehicle | 0 | 0 | 0 |
| ADC-1 (10 mg/kg) (GB01 Exatecan) | 0 | 3 | 3 |
| ADC-4 (10 mg/kg) (B12 Exatecan) | 0 | 0 | 0 |

Tables 10 to 12 show Mean Tumor Volume mm³ at days 0 and 42 (mean of n=3-5)

TABLE 10
In vivo anti-tumour activity in LU2049 model

| Response summary | Day 0 | Day 42 | % change |
|---|---|---|---|
| Vehicle | 152.49 | 572.09 | +275.17% |
| ADC-1 (10 mg/kg) (GB01 Exatecan) | 152.73 | 212.39 | +39.06% |
| ADC-4 (10 mg/kg) (B12 Exatecan) | 152.49 | 449.56 | +194.81% |

TABLE 11
In vivo anti-tumour activity in LU1144 model

| Response summary | Day 0 | Day 42 | % change |
|---|---|---|---|
| Vehicle | 163.55 | 655.81 | +300.98% |
| ADC-1 (10 mg/kg) (GB01 Exatecan) | 162.59 | 307.32 | +89.02% |
| ADC-4 (10 mg/kg) (B12 Exatecan) | 163.40 | 671.12 | +310.72% |

TABLE 12
In vivo anti-tumour activity in LU11836 model

| Response summary | Day 0 | Day 42 | % change |
|---|---|---|---|
| Vehicle | 134.51 | 608.82 | +352.62% |
| ADC-1 (10 mg/kg) (GB01 Exatecan) | 133.71 | 0 | −100% |
| ADC-4 (10 mg/kg) (B12 Exatecan) | 132.67 | 107.39 | −19.05% |

### Conclusions

The GB01 Exatecan ADC was more efficacious than B12 Exatecan (isotype control ADC) across the panel of Lung PDXs.

A number of publications are cited above to more fully describe and disclose the disclosures and the state of the art to which inventions herein may pertain. The entirety of each of the references mentioned in this disclosure are hereby is incorporated by reference.

1. An antibody drug conjugate of formula (I):
Ab-L-Dp (1)
wherein:
Ab is an antibody that binds to Claudin-6, which antibody comprises (i) an immunoglobulin heavy chain variable region having a CDR1 region with the amino acid sequence shown in SEQ ID NO: 3, a CDR2 region with the amino acid sequence shown in SEQ ID NO: 4, and a CDR3 region with the amino acid sequence shown in SEQ ID NO: 5; and (ii) an immunoglobulin light chain variable region having a CDR1 region with the amino acid sequence shown in SEQ ID NO: 6, a CDR2 region with the amino acid sequence shown in SEQ ID NO: 7, and a CDR3 region with the amino acid sequence shown in SEQ ID NO: 8;
L-Dp is a drug linker conjugated to the Ab wherein L comprises a polyglycine moiety, Glyₙ wherein n is from 5 to 8, and D is an Exatecan, wherein the drug linker can be cleaved under cellular conditions to release an Exatecan with the following formula (II): 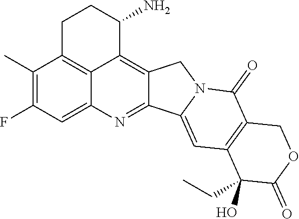 [SMILES: CC[C@@]1(O)C(=O)OCc2c1cc1n(c2=O)Cc2c-1nc1cc(F)c(C)c3c1c2[C@@H](N)CC3]
and wherein p is the number of drug units per antibody and is from 1 to 6.

2. A conjugate according to claim 1 wherein the antibody has a VH domain as shown in SEQ ID NO: 1, and a VL domain as shown in SEQ ID NO: 2.

3. A conjugate according to claim 1 wherein the antibody has a heavy chain as shown in SEQ ID NO: 9 or 10. and a light chain as shown in SEQ ID NO: 11 or 12.

4. A conjugate according to claim 1 wherein the drug linker is conjugated to the Ab via one or more cysteine residues and Ab has a mutation in one, two or three hinge region cysteines.

5. A conjugate according to claim 4 wherein Ab has a mutation in Cysteine 226 (EU numbering) of the heavy chain, such as a Cys to Valine substitution.

6. A conjugate according to claim 4 wherein Ab has an engineered cysteine outside the hinge region, such as in the light chain, such as a V205C mutation.

7. A conjugate according to claim 6 wherein Ab comprises the following heavy chain mutations: a Leucine 234 to Alanine substitution, a Leucine 235 to Alanine substitution and a Proline 329 to Alanine substitution (EU numbering).

8. A conjugate according to claim 1 wherein the linker comprises a cathepsin cleavable sequence e.g. Val-Ala or Val-Cit.

9. A conjugate according to claim 1 wherein the linker comprises a self-immolative moiety operably linked to the Exatecan, such as para-aminobenzylcarbamate.

10. A conjugate according to claim 1 where L-Dp is:

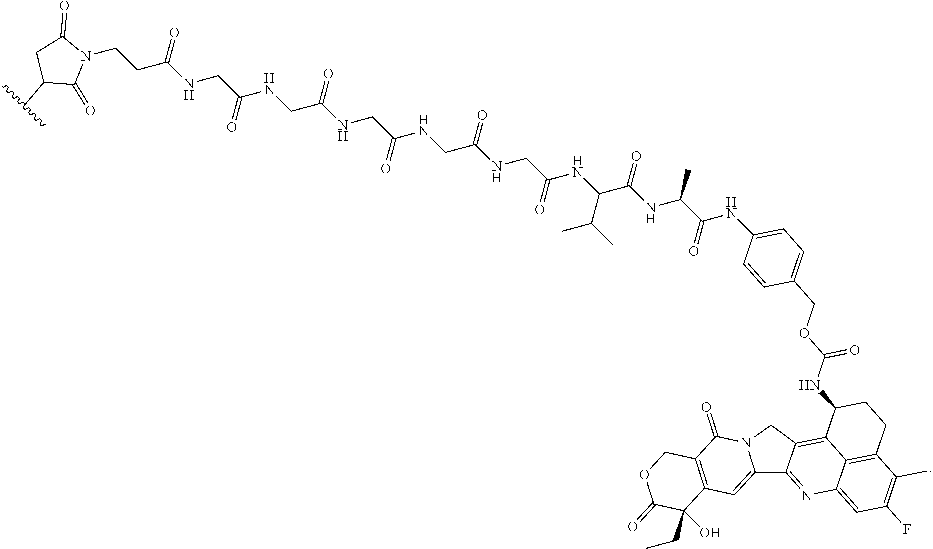 [SMILES: *C1CC(=O)N(CCC(=O)NCC(=O)NCC(=O)NCC(=O)NCC(=O)NCC(=O)NC(C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

11. A conjugate according to claim 2 where L-Dp is:

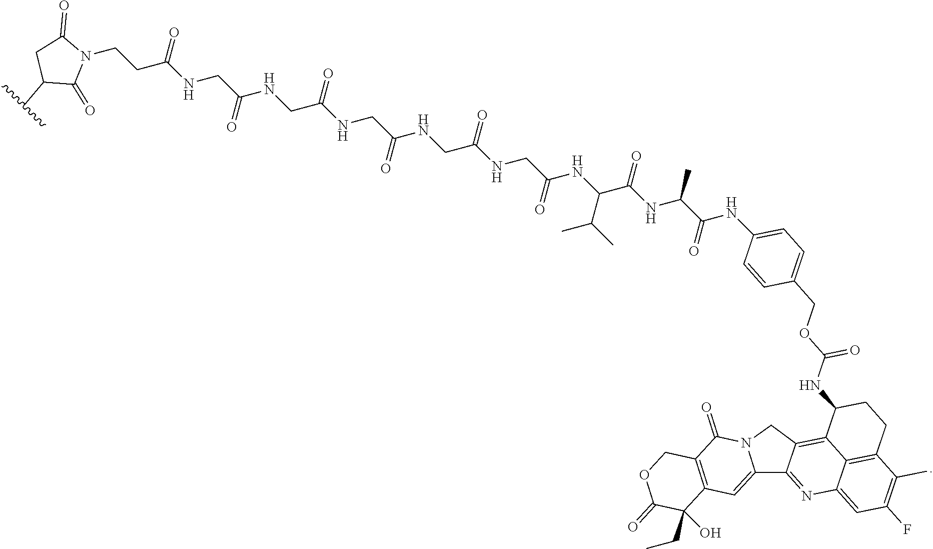 [SMILES: *C1CC(=O)N(CCC(=O)NCC(=O)NCC(=O)NCC(=O)NCC(=O)NCC(=O)NC(C(=O)N[C@@H](C)C(=O)Nc2ccc(COC(=O)N[C@H]3CCc4c(C)c(F)cc5nc6c(c3c45)Cn3c-6cc4c(c3=O)COC(=O)[C@]4(O)CC)cc2)C(C)C)C1=O]

12. A composition comprising a mixture of antibody drug conjugates according to claim 1 wherein the average drug to antibody ratio is from 3 to 6.

13. A method of treating an individual suffering from a proliferative disease selected from ovarian cancer, non-small cell lung carcinoma (NSCLC), gastric cancer, oesophageal cancer, endometrial cancer and hepatocellular carcinoma (HCC), which method comprises administering to the patient a conjugate according to claim 1.


SEQUENCE LISTING (22 sequences)

SEQ ID NO: 1 - protein, 116 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSS

SEQ ID NO: 2 - protein, 113 aa, synthetic construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIK

SEQ ID NO: 3 - protein, 6 aa, Mus musculus
SGYYWN

SEQ ID NO: 4 - protein, 16 aa, Mus musculus
YISYSGSNNYNPSLKN

SEQ ID NO: 5 - protein, 7 aa, Mus musculus
GGYYFDY

SEQ ID NO: 6 - protein, 17 aa, Mus musculus
KSSQSLLNTNNQKNYLA

SEQ ID NO: 7 - protein, 7 aa, Mus musculus
FASTRES

SEQ ID NO: 8 - protein, 9 aa, Mus musculus
QQHYNAPRT

SEQ ID NO: 9 - protein, 446 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
QVQLQESGPGLVKPSETLSLTCTVSGYSITSGYYWNWIRQPPGKGLEWMGYISYSGSNNYNPSLKNRVTISRDTSKNQFSLKLSSVTAADTAVYYCARGGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 220 aa, synthetic construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 220 aa, synthetic construct
DIVMTQSPDSLAVSLGERATINCKSSQSLLNTNNQKNYLAWYQQKPGQPPKLLIYFASTRESGVPDRFSGSGSGTDFTLTISSLQAEDVAVYFCQQHYNAPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 13 - protein, 451 aa, synthetic construct
EVQLVESGGGLVQPGGSLRLSCAASGFTFSIYGMSWVRQAPGKGLEWVATISRDASYTYFPDSVKGRFTISRDNAKNSLYLQMNSLRAEDTAVYYCVRLGDNDRGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 219 aa, synthetic construct
DVVMTQSPLSLPVTLGQPASISCRSSQSVVHVNANTYLEWYQQRPGQSPRLLIYKVSNRFPGVPDRFSGSGSGTDFTLKISRVEAEDVGVYYCFQGSHVPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 4 aa, synthetic construct
GGFG

SEQ ID NO: 16 - protein, 5 aa, synthetic construct
GGGGG

SEQ ID NO: 17 - protein, 6 aa, synthetic construct
GGGGGG

SEQ ID NO: 18 - protein, 7 aa, synthetic construct
GGGGGGG

SEQ ID NO: 19 - protein, 8 aa, synthetic construct
GGGGGGGG

SEQ ID NO: 20 - protein, 5 aa, synthetic construct
  REPEAT 5: note = Glycine at position 5 may be repeated 1 to 3 times to give a range of 5 to 8 glycine residues in total
GGGGG

SEQ ID NO: 21 - protein, 7 aa, synthetic construct
GGGGGVA

SEQ ID NO: 22 - protein, 5 aa, synthetic construct
GGGVA